(19) United States
(12) Patent Application Publication
(10) Pub. No.: US 2024/0082451 A1
(43) Pub. Date: Mar. 14, 2024

(54) DEVICE FOR DIFFUSING VOLATILE SUBSTANCES

(71) Applicant: ZOBELE Holding S.p.A., Trento (IT)

(72) Inventors: Marina TRIAS LAFUENTE, Barcelona (ES); Moisés CABALLERO TAPIA, Barcelona (ES); Fernando ALFONSO GALLEGO, Barcelona (ES)

(73) Assignee: ZOBELE Holding S.p.A., Trento (IT)

(21) Appl. No.: 17/766,741

(22) PCT Filed: Oct. 8, 2020
(86) PCT No.: PCT/EP2020/078271

§ 371 Date: Apr. 6, 2022

(30) Foreign Application Priority Data
Oct. 9, 2019 (ES) P201930883

(51) Int. Cl. A61L 9/12

(52) U.S. Cl. CPC A61L 9/127; A61L 2209/133

(57) ABSTRACT

The device for diffusing volatile substances comprises a container (1) containing a liquid with said volatile substances, a wick (2) that is impregnated with said volatile substances, and also comprises a support element (3) that supports the wick (2) in a removably manner.

It allows providing a volatile substances device that allows the reuse of this type of devices provided with a wick.

The present invention relates to a device for the diffusion of volatile substances.

### BACKGROUND OF THE INVENTION

There are many types of air fresheners on the market, one of which consists of static air fresheners.

These static air fresheners consist of the use of a large surface wick that is constantly in contact with a liquid containing volatile substances, so that said liquid permeates the wick, which diffuses the volatile substances into the environment.

The main drawback of these static devices is that they are for single use, since if they were reused it would force the user to come into contact with the wick impregnated with the liquid containing volatile substances.

Therefore, an object of the present invention is to provide a device for volatile substances that allows the reuse of this type of devices provided with a wick.

### DESCRIPTION OF THE INVENTION

With the device for diffusing volatile substances according to the invention, the aforementioned drawbacks are solved, presenting other advantages that will be described below.

The device for diffusing volatile substances according to the present invention comprises:
a container containing a liquid with such volatile substances,
a wick that is impregnated with said volatile substances, wherein the diffusion device also comprises a support element that supports the wick in a removable manner.

Advantageously, said support element comprises a guide for positioning the wick.

Furthermore, said support element preferably also comprises a cover to which the wick is removably fixed, which facilitates the removal and placement of the wick from/on the support element.

Said cover advantageously comprises a groove in which a complementary projection of said wick is housed.

According to a preferred embodiment, the wick comprises a first and a second portion, the first portion being wider than the second portion, and said protrusion is preferably located in said first portion.

Preferably, said support member comprises a plurality of evaporation holes and a cover portion surrounding the part of the wick housed inside the container, said cover portion comprising a plurality of through holes.

If desired, the wick can comprise a handle that facilitates the handling of the wick and, in this case, the projection is preferably arranged on said handle.

The device for diffusing volatile substances according to the present invention has at least the following advantages:
It allows the replacement of the wick without the need for the user to come into contact with the impregnated wick,
It ensures correct assembly,
It ensures correct dosage and evaporation of volatile substances,
It allows the reuse of parts.

### BRIEF DESCRIPTION OF THE DRAWINGS

For a better understanding what has been stated, some drawings are attached in which, schematically and only as a non-limiting example, a practical case of embodiment is represented.

FIG. 1 is an exploded perspective view of the device for diffusing volatile substances according to the present invention.

### DESCRIPTION OF A PREFERRED EMBODIMENT

As shown in the FIGURES, the device for diffusing volatile substances comprises a container 1 containing a liquid with the volatile substances.

In said container 1 a wick 2 is placed, which is impregnated with the liquid, whose volatile substances diffuse into the environment.

The wick 2, according to the embodiment shown, comprises first and second portions 22, 23, the first portion 22 being wider than the second portion 23.

According to the illustrated embodiment, said first portion 22 is the upper portion and the second portion 23 is the lower portion.

The diffusion device according to the present invention also comprises a support element 3, the function of which is to hold the wick 2 in the correct position of use.

For this, the support element 3 comprises a guide 32 for the wick 2. Said guide is formed by profiles parallel to each other, among which the wick 2 is housed.

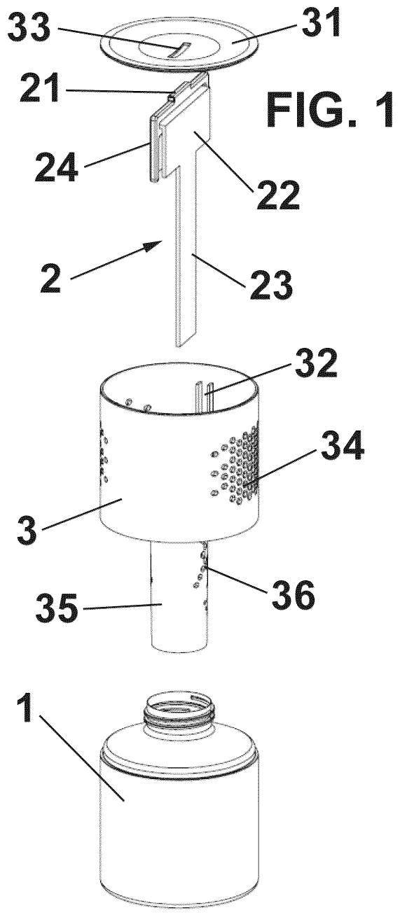

In FIG. 1 the upper ends of two of said profiles can be seen, and in an opposite position there are two identical profiles.

To manipulate the wick 2, that is to say, for its placement and removal, the wick 2 preferably comprises a handle 24, which is attached to the first portion 22 of the wick 2. Preferably, said handle 24 is made of a waterproof material, so it will not be impregnated with volatile substances.

Said handle 24 comprises a projection 21 that is housed in a complementary groove 33 of a cover 31 of said support element 3.

It should be noted, however, that said handle 24 is not essential, since the projection 21 could be arranged on the wick 2 itself, in particular, in the first portion 22, the wick 2 being manipulated by means of said cover 31

Despite the fact that reference has been made to a specific embodiment of the invention, it is obvious to a person skilled in the art that the device described for diffusing volatile substances is susceptible to numerous variations and modifications, and that all the mentioned details can be substituted by others technically equivalent, without departing from the scope of protection defined by the appended claims.

1. Device for diffusing volatile substances, comprising:
a container (1) containing a liquid with said volatile substances,
a wick (2) that is impregnated with said volatile substances,
characterized in that it also comprises a support element (3) that supports the wick (2) in a removable manner.

2. Device for diffusing volatile substances according to claim 1, wherein said support element (3) comprises a guide (32) for positioning the wick (2).

3. Device for diffusing volatile substances according to claim 1, wherein said support element (3) comprises a cover (31) to which the wick (2) is removably fixed.

4. Device for diffusing volatile substances according to claim 3, wherein said cover (31) comprises a groove (33) in which a complementary projection (21) of said wick (2) is housed.

5. Device for diffusing volatile substances according to claim 1, wherein the wick (2) comprises first and second portions (22, 23), the first portion (22) being wider than the second portion (23).

6. Device for diffusing volatile substances according to claim 4, wherein said projection (21) is positioned on said first portion (22).

7. Device for diffusing volatile substances according to claim 1, wherein said support element (3) comprises a plurality of evaporation holes (34).

8. Device for diffusing volatile substances according to claim 1, wherein said support element (3) comprises a cover portion (35) that surrounds the part of the wick (2) housed inside the container (1).

9. Device for diffusing volatile substance according to claim 8, wherein said cover portion (35) comprises a plurality of through holes (36).

10. Device for diffusing volatile substances according to claim 1, wherein the wick (2) comprises a handle (24).

11. Device for diffusing volatile substance according to claim 4, wherein the projection (21) is arranged on said handle (24).
